(19) United States
(12) Patent Application Publication
(10) Pub. No.: US 2023/0287072 A1
(43) Pub. Date: Sep. 14, 2023

(54) COMPOSITION AND METHOD FOR TREATING NEUROPATHY

(71) Applicants: The Regents of the University of California, Oakland, CA (US); The Steadman Clinic & Steadman Philippon Research Institute, Vail, CO (US)

(72) Inventors: Chengbiao WU, La Jolla, CA (US); Kijung SUNG, La Jolla, CA (US); Chelsea Shileds BAHNEY, Edwards, CO (US); Kevin RIVERA, San Francisco, CA (US); Tejal DESAI, San Francisco, CA (US)

(73) Assignees: The Regents of the University of California, Oakland, CA (US); The Steadman Clinic & Steadman Philippon Research Institute, Vail, CO (US)

(21) Appl. No.: 18/019,160

(22) PCT Filed: Aug. 2, 2021
(86) PCT No.: PCT/US2021/044174

§ 371 Date: Feb. 1, 2023

Related U.S. Application Data
(60) Provisional application No. 63/060,872, filed on Aug. 4, 2020.

(51) Int. Cl. C07K 14/48; A61K 9/00; B82Y 30/00; A61K 47/69; A61P 25/02

(52) U.S. Cl. CPC C07K 14/48; A61K 9/0092; B82Y 30/00; A61K 47/6935; A61P 25/02

(57) ABSTRACT

The present disclosure provides a method of delivery, treatment, and prevention of neuropathy and/or pain associated with NGF treatment for an underline disease or condition with a NGF mutant, such as NGF^{R100W}, that does not elicit pain. The present disclosure further provides a composition of micro- and/or nano-rods attached with the NGF mutant, such as NGF^{R100W}, which are injectable or administered to a target for desired therapies.

### CROSS-REFERENCE TO RELATED APPLICATIONS

This PCT international application claims the benefit of U.S. Provisional Application No. 63/060,872, filed Aug. 4, 2020, which is incorporated herein by reference in its entirety.

### FIELD OF INVENTION

The present disclosure relates generally to nerve growth factor (NGF) and its mutant(s) that could be used for treating neuropathy and pain associated with the NGF treatment.

### BACKGROUND OF THE INVENTION

NGF is a member of the neurotrophic factor family that provides potent trophic actions on sensory and sympathetic neurons of the peripheral nervous system (PNS) (Chao, 2003; Chao and Bothwell, 2002; Chao and Hempstead, 1995; Chen et al., 2008; Huang and Reichardt, 2001; Levi-Montalcini and Hamburger, 1951). NGF also regulates the trophic status of striatal and basal forebrain cholinergic neurons (BFCNs) of the central nervous system (CNS) (Conover and Yancopoulos, 1997; Kew et al., 1996; Lehmann et al., 1999; Levi-Montalcini and Hamburger, 1951; Li and Jope, 1995; Svendsen et al., 1994). BFCNs play a critical role in learning and memory through regulating the excitability of the hippocampus and neocortex (Deiana et al., 2011; Drachman and Leavitt, 1974; Dutar et al., 1995; Everitt and Robbins, 1997). Degeneration of BFCNs/entorhinal cortex occurs in early phases of Alzheimer's disease (AD) (Davies and Maloney, 1976; Grothe et al., 2012; Ward et al., 2000; Whitehouse et al., 1982), which has provided support for the cholinergic hypothesis in AD (Bartus et al., 1982; Francis et al., 1999). Acetylcholinesterase inhibitors that preserve cholinergic function have thus been developed for symptomatic treatment of AD (Doody, 2003; Grutzendler and Morris, 2001; Wilkinson et al., 2004). NGF, derived from hippocampus and cortex, provides robust trophic support for BFCNs for their survival, differentiation and functional maintenance (Conner et al., 2009; Cuello et al., 2007; Fischer et al., 1987; Gnahn et al., 1983; Holtzman et al., 1992; Houeland et al., 2010; Latina et al., 2017; Levi-Montalcini, 1987; Niewiadomska et al., 2011). Degeneration of BFCNs in AD points to a deficit in NGF support. Consistent with this notion, deficiency in production, trafficking and signaling of NGF has been shown to induce selective and extensive atrophy of BFCNs (Capsoni et al., 2000; Cooper et al., 2001; Counts and Mufson, 2005; Delcroix et al., 2004; Salehi et al., 2004; Schliebs and Arendt, 2006; Schmitz and Nathan Spreng, 2016; Sofroniew et al., 1993; Teipel et al., 2014). Based on these findings, NGF replacement has emerged as a disease-modifying treatment strategy in mild AD patients (Cattaneo and Calissano, 2012; Mufson et al., 1995; Tuszynski and Gage, 1995; Tuszynski et al., 2005; Tuszynski et al., 2015). In an early clinical test, AD patients were treated by intracerebroventricular (ICV) infusion of NGF (Eriksdotter Jonhagen et al., 1998). However, the trial was terminated due to intolerable back pain (Aloe et al., 2012; Eriksdotter Jonhagen et al., 1998; Olson, 1993).

Strong preclinical and clinical data also links reduced NGF to pathogenesis of neuropathies such as diabetic polyneuropathy (DPN). NGF levels were decreased in diabetic rats induced by streptozocin (Hellweg and Hartung, 1990); NGF level was low in serum and submaxillary glands of diabetic mice (Ordonez et al., 1994); Retrograde delivery of NGF was also diminished in diabetic rodents (Jakobsen et al., 1981); Serum NGF level was found to be significantly lower in patients of severe diabetic neuropathy (Faradji V, Sotelo J: Low serum levels of nerve growth tactor in diabetic neuropathy. Acta Neurol Scand. 1990, 81: 402-406) suggesting that administration of NGF could serve as an effective treatment for DPN. Recombinant human NGF (rhNGF) was tested at two dosages (0.1, 0.5 μg/kg) in clinical trials for treating DPN in 1994 (Apfel, 1999; Apfel and Kessler, 1996; Apfel et al., 1998). The trial showed some clinical benefits in Phase II. But out of concern of significant pain associated with the 0.5 μg/kg dose, only was the 0.1 μg/kg dose selected for Phase Ill. The results did not show significant clinical benefits. Pain induced by higher doses of NGF was likely the primary reason for terminating the clinical efforts (Apfel, 2002). Therefore, defining the signaling mechanisms underlying NGF^{R100W} could enhance the fundamental understandings of NGF biology and reignite efforts to finally realize the therapeutic potentials of NGF.

A point missense recessive mutation in NGF was discovered in Swedish patients who suffered from severe loss of deep pain, bone fractures and joint destruction (Carvalho et al., 2011; Einarsdottir et al., 2004a, b; Enocson et al., 2006; Minde et al., 2009; Minde et al., 2006; Minde et al., 2004; Minde, 2006; Perini et al., 2016; Sagafos et al., 2016). This rare disorder has been classified HSAN V. Genetic analysis has identified a point mutation that changed a highly conserved arginine (R) to tryptophan (W) at the 100 position in mature NGF (NGF^{R100W}) (Einarsdottir et al., 2004b). Interestingly, these patients appeared to have normal cognitive function (Einarsdottir et al., 2004a; Minde et al., 2004; Minde, 2006; Morrison et al., 2011; Perini et al., 2016), suggesting that NGF^{R100W} retains its trophic function without inducing nociception (Einarsdottir et al., 2004b; Morrison et al., 2011). This is different from the other two known NGF mutations NGF^{R121W} (Shaikh et al., 2018) and NGF[^{680C>A]+[681_682delGG]} (Garvalho et al., 2011) that are also linked to HASN V. In both cases patients lost pain perception but also suffered from cognitive disabilities (Carvalho et al., 2011; Shaikh et al., 2018). Therefore, NGF^{R100W} is uniquely interesting in that the pain causing effect is likely de-coupled from its trophic function.

### SUMMARY OF THE INVENTION

The present disclosure provides a unique micro- or nano-rods comprising a painless NGF mutant, and a method of use thereof, to deliver the painless NGF mutant to a target for therapies, particularly for treating neuropathy and/or pain associated with the NGF treatment. In certain embodiments, the present disclosure provides micro- or nano-rods attached with the painless NGF mutant, and a composition of packaging and delivery methods with the painless NGF mutant. More particularly, the painless NGF mutant is packaged into micro- or nano-rods, and such packaged micro- or nano-rods attached with the painless NGF mutant is embedded under the skin for of subject in need, e.g., a human patient, who suffers with neuropathy and/or pain associated with the NGF treatment. The micro- or nano-rods control the release of the painless NGF mutant, that even at high dose won't cause intolerable pain. The painless NGF mutant promotes the growth of nerve fibers that are damages in neuropathy.

The present disclosure thus provides a method of treating neuropathy and/or pain associated with the NGF therapies by administering a composition of micro- or nano-rods attached with a painless NGF mutant. In certain embodiments, the painless NGF mutant is NGF^{R100W}. The peripheral neuropathies are common in diabetic patients, HIV-infected patients and in patients receiving chemotherapy drugs.

In certain embodiments, the poly-caprolactone (PCL) nanowire platform technology is functionalized for bioactivity through the attachment of the painless NGF mutant using a Layer-by-Layer (LbL) electrostatic assembly approach. The PCL nanowires bear a strong negative charge that can be capitalized upon to electrostatically assemble chitosan (positive charge) and heparin (negative charge). Chitosan has antimicrobial properties towards both gram negative and gram positive bacteria and has been used successfully in medical device coatings and drug delivery systems. Heparin was chosen for its ability to bind to and stabilize a variety of growth factors with moderate to high affinities and serves a modular means of loading growth factor cargo onto these nanowires.

Importantly, these nanowires are designed to enable a point-of-care delivery technology with high clinical relevance. Nanowires maintain their structure and function following lyophilization (freeze drying), which creates a drug delivery platform with increased stability. To deliver the nanorods, they simply and quickly rehydrate in PBS and then can be injected percutaneously into a patient in need and/or embedded under the skin of a patient in need. The PCL nanowires are 15-20 nm in length and therefore do not interfere with normal healing or treatment. In addition to PCL, hydrogels and liposomes can also be used to make the micro- and/or nano-rods.

The present disclosure further provides the dose limits of NGF^{R100W} in eliciting pain in vivo. In addition, the present disclosure also provides that NGF^{R100W} rescues small sensory nerve fiber degeneration both structurally and functionally, thus, providing significant insights into the translational potential of NGF^{R100W}.

### BRIEF DESCRIPTION OF THE DRAWINGS

FIGS. 1A-1B. Intraplantar injection of NGF^{R100W} does not induce sensitization. The hind paws (n=6) were injected with 0.6 μg of either wtNGF or NGF^{R100W} and thermal threshold was measured (FIG. 1A). In FIG. 1B, 0.2 μg of wtNGF or NGF^{R100W} was injected into the rat's hind paws (n=6) mechanical threshold was measured. Adult WT mice (3 mo old) were injected with 0.5 μg NGF^{WT} (n=4) or NGF^{R100W} at 0.5 μg (n=4), 1.0 μg (n=5), 5.0 μg (n=4) at indicated doses and hotplate (55° C.) assays were performed 20 min after injection. Paw withdraw latencies (sec) were recorded (FIG. 1A). In FIG. 1B decreased thermal thresholds were presented. Mean±SEM, Two-Way ANOVA with Bonferroni post-test.

FIGS. 2A-2C. A CMT2B Rab7^{V162M} knockin mouse model (C57BL6). FIG. 2A: a schematic showing the knockin strategy. FIG. 2B: PCR primers: primer 1 (Rab7-loxPtF2) and primer 2 (Rab7-loxPtR2) and tail DNA genotyping results of wt (+/+), heterozygote (fln/+) and homozygote (fln/fln). FIG. 2C: both fln/+, fln/fln mice survived to adulthood.

FIGS. 3A-3G: Footpad Histopathology (FIG. 3A-3F) Immunolabeling of intraepidermal nerve fibers (white arrows) in footpad biopsies of 9-month-old Rab7V162M mice. Nerve fibers stained for PGP 9.5 shown in red. Nuclei are stained with DAPI shown in blue. De, dermis; epi, epidermis. Dotted white lines denote dermo-epidermal boundaries. Solid white lines denote skin surface. (FIG. 3G) Relative fluorescence signal intensity was quantified using NIH ImageJ. Data are mean±SEM, n=3 for each group. *=p<0.05, **=p<0.01 vs control (+/+) by ANOVA with Dunnett's test.

FIG. 4: 9 month old CMT2B peripheral sensory neuropathy mutant mice were injected intradermally with either 1 μg of wtNGF or 1 μg of NGF^{R100W} once a week and for a total of 6 weeks. Thermal pain tests (55° C.) were conducted “before” and “after” the duration. Skin sections from hind paws were also analyzed for the status of sensory fiber regeneration.

FIG. 5. NGF^{R100W} is effective in restoring peripheral sensory function in CMT2B mutant mice. Hot plate tests (55° C.) were conducted ‘before’ and ‘after injection’ into 9 mo-old CMT2B mutant mice of either 1 μg of wtNGF or 1 μg of NGF^{R100W} once a week and for a total of 6 weeks. 9 mo-old WT littermates were used as comparison. The response latencies of ‘before’ and ‘after injection’ were measured and compared. Mean±SEM, *** p<0.01, One Way ANOVA (Kruskal-Wallies test).

FIGS. 6A-6F: CMT2B mutant mice were injected intradermally with either wtNGF (FIGS. 6B & 6E) or NGF^{R100W} (FIGS. 6C & 6F) or untreated (FIG. 6A) as in FIG. 5. Mice were sacrificed and hindpaw skins were extracted, fixed, sectioned and stained for PGP9.5 as described in our recent publication Prog Neurobiol. 2020 November; 194:101886. doi: 10.1016/j.pneurobio.2020.101886. Epub 2020 Jul. 18. PubMed PMID: 32693191. Epi: epidermis; SNP: subepidermal neural plexus; red arrow heads: IENF (intra-epidermal nerve fibers). IENF density was quantitated and shown in FIG. 6D. One-Way ANOVA Mean±SEM, *** p<0.005; **** p<0.001 (Dunnett's test), n=3.

### DETAILED DESCRIPTION OF THE INVENTION

The present disclosure provides a novel method for treating neuropathy using a NGF mutant. In certain embodiments the NGF mutant, NGF^{R100W}, used as an effective therapy for treating peripheral sensory neuropathy. In certain embodiments, the NGF^{R100W} is injected at 10× of normal wildtype NGF without inducing significant sensitization, suggesting that NGF^{R100W} is as efficacious as the wildtype NGF in promoting the regrowth of small sensory fibers and rescuing functional degeneration in a mouse model of peripheral sensory neuropathy.

The following description of the embodiments is merely exemplary in nature and is in no way intended to limit the present disclosure, its application, or uses.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a short chain fatty acid,” “a carnitine derivative,” or “an adjuvant,” includes, but is not limited to, combinations of two or more such short chain fatty acids, carnitine derivatives, or adjuvants, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a short chain fatty acid refers to an amount that is sufficient to achieve the desired improvement in the property modulated by the formulation component, e.g. achieving the desired level of reduction of withdrawal symptoms. The specific level in terms of wt % in a composition required as an effective amount will depend upon a variety of factors including the amount and type of short chain fatty acid, amount and type of carnitine derivative, amount and type of pharmaceutically acceptable excipients, and disorder being treated using the disclosed compositions.

As used herein, the terms “optional” or “optionally” means that the subsequently described event or circumstance can or cannot occur, and that the description includes instances where said event or circumstance occurs and instances where it does not.

As used herein, “treatment” or “treating” is an approach for obtaining beneficial or desired results including clinical results. For purposes of the present disclosure, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of the disease. The methods provided herein contemplate any one or more of these aspects of treatment.

As used herein, “patient” includes human or non-human (i.e., animal) patient. In a particular embodiment, the invention encompasses both human and nonhuman. In another embodiment, the invention encompasses nonhuman. In another embodiment, the term encompasses human.

Animal: As used herein, the term “animal” refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In certain embodiments, the animal is susceptible to infection by influenza. In some embodiments, an animal may be a transgenic animal, genetically engineered animal, and/or a clone.

As used herein, “therapeutically effective amount” refers to an amount effective, when administered to a human or non-human patient, to provide a therapeutic benefit such as amelioration of symptoms, slowing of disease progression, or prevention of disease. The specific dose of substance administered to obtain a therapeutic benefit will, of course, be determined by the particular circumstances surrounding the case, including, for example, the specific substance administered, the route of administration, the condition being treated, and the individual being treated.

In one embodiment, provided herein is a composition and method for treating e.g., neuropathy using a NGF mutant, e.g., NFG^{R100W}. As used herein, “neuropathy” and/or “peripheral neuropathy” can be used interchangeable which refers to disease or dysfunction of one or more peripheral nerves, typically causing numbness, weakness, and pain from nerve damages, usually in the hands and feet. A common cause of peripheral neuropathy includes but is not limited to diabetes (diabetic neuropathy), HIV (HIV-induced neuropathy), chemotherapy (CHEMO-induced neuropathy), injuries, infections, and exposure to toxins. Methods for diagnosis and measurement of neuropathy are well known in the art. In certain embodiments of treating neuropathy, the effective treatment is measure by the level of numbness, weakness, and/or pain that is reduced by about 5% to about 100%. In one embodiment, the level of numbness, weakness, and/or pain is reduced by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, or about 100% in the subject.

### Method for Delivery, Treatment & Prevention

In certain embodiments, the present disclosure provides a method for treating a neuropathy comprising administering to a subject in need an effective amount of a composition comprising an effective amount of a NGF mutant, such as NGF^{R100W}, that does not elicit pain. In certain embodiments, the composition is packaged in micro-rods and/or nano-rods that deliver the NGF mutant, such as NGF^{R100W}. The neuropathy may be caused by diabetes, HIV, Chemotherapy, infections, injuries, or exposure to a toxin. The subject in need is a human being and/or non-human animals who suffers with neuropathy.

In certain embodiments, the methods provided herein comprise administering a pharmaceutical composition comprising the NGF mutant, such as NGF^{R100W}, of the present disclosure and a pharmaceutically acceptable carrier or excipient. Combination formulations and/or treatment according to the present disclosure comprise the NGF mutant of the present disclosure together with one or more pharmaceutically acceptable carriers or excipients and optionally other therapeutic agents, now known or later developed, for treating and/or preventing neuropathy and its associated conditions. Combination formulations containing the active ingredient may be in any form suitable for the intended method of administration. In certain embodiments, the pharmaceutical composition comprising a desired dose of NGF^{R100W} in micro- and/or nano-rod is injected to a subject in need.

In certain embodiments, the poly-caprolactone (PCL) nanowire platform technology is functionalized for bioactivity through the attachment of NGF^{R100W} using a Layer-by-Layer (LbL) electrostatic assembly approach, as previously published (Zamecnik et al., 2017), the entire content of which is incorporated by reference herein. The PCL nanowires bear a strong negative charge that is capitalized upon to electrostatically assemble chitosan (positive charge) and heparin (negative charge) onto the nanowires. In addition to its positive charge, chitosan has antimicrobial properties and therefore has been used successfully in medical device coatings and drug delivery systems (Pant et al., 2019; Perinelli et al., 2018; Rabea et al., 2003). Heparin was chosen for its ability to bind to and stabilize a variety of growth factors, including NGF, with moderate to high affinities and serves a modular means of loading growth factor cargo onto nanowires. (Jeon et al., 2011; Jha et al., 2015; Rabenstein, 2002; Rindone et al., 2019; Xu et al., 2011; Zhang et al., 2017).

In certain embodiments, to attach the NGF mutant, such as NGF^{R100W} cargo, to nanowires, a layer-by-layer (LbL) electrostatic assembly approach is utilized according to Zamecnik et al., 2017. LbL assembly has been used extensively for drug delivery applications and affords a facile and modular means of attaching biological cargo onto nanomaterials, such that increasing layers increase growth factor retention (Woodruff and Hutmacher, 2010; Xue et al., 2013; Zamecnik et al., 2017). The PCL nanowires bear a strong negative charge as a result of the alkaline etching method used in the fabrication process. This negative charge to electrostatically assemble biopolymers onto the surface of the nanowires. Chitosan (positive charge) and heparin (negative charge) were chosen for LbL assembly due to their biocompatibility and the growth factor affinity of heparin. Both chitosan and heparin have been successfully deposited onto the surface of the nanowires, as determined by zeta potential measurements of nanowire surface charge. Multiple layers were deposited, resulting in observed charge oscillation between positively charged (chitosan), and negatively charged (heparin-coated) nanowires.

Importantly, these nanowires are designed to enable a non-surgical delivery technology with high clinical relevance. In certain embodiments, PCL nanowires are 200 nm in diameter and 15-20 nm in length. Due to their small size they can be easily injected for percutaneous delivery to the desired sight and will not interfere with the normal healing process as some current materials have been shown to do (McConoughey et al., 2015). Besides, PCL, hydrogels, liposomes can also be used for making the nano-rods.

In other embodiments, the present disclosure also provides a method of treating or preventing pain induced by a treatment with a NGF for an underline disease or condition, comprising administering to a subject in need an effective amount of a composition comprising an effective amount of a NGF mutant, such as NGF^{R100W}, that does not elicit pain, alone or in conjunction with the NGF treatment for the underline disease and/or condition. In certain embodiments, the effective amount of NGF mutant, such as NGF^{R100W}, can be 2-10 times, preferably, 2×, 3×, 4×, 5×, 6×, 7×, 8×, 9×, and 10× more than the effective amount of NGF for the underline disease or condition. The effective amount of the NGF mutant, such as NGF^{R100W} is packaged in micro-rods and/or nano-rods for delivery of the NGF^{R100W} into the desired nerve and/or tissue. In certain embodiments, the nano-rods uses poly-caprolactone (PCL) nanowire platform functionalized for bioactivity through the attachment of the NGF mutant, such as NGF^{R100W}, by a layer-by-layer (LbL) electrostatic assembly approach. In other embodiments, hydrogels and/or liposomes can also be used for making the nano-rods for delivering the NGF mutant, such as NGF^{R100W}

### Pharmaceutical Compositions, Dose & Administration

In certain embodiments, the present disclosure provides the pharmaceutical compositions comprising an effective amount of a NGF mutant, such as NGF^{R100W}. In certain embodiments, such pharmaceutical composition can be formulated in a suitable formulation according to any conventional method and be administered via any suitable administration route. In certain embodiments, such pharmaceutical composition is micro- or nano-rod comprising the NGF mutant, such as NGF^{R100W}, that are injectable and tissue-specific.

While it is possible for an active ingredient to be administered alone, it may be preferable to present them as pharmaceutical formulations or pharmaceutical compositions as described above. The formulations, both for veterinary/animal and for human use, of the disclosure comprise at least one of the active ingredients, together with one or more acceptable carriers therefor and optionally other therapeutic ingredients. The carriers must be “acceptable” in the sense of being compatible with the other ingredients of the formulation and physiologically innocuous to the recipient thereof.

Each of the active ingredients can be formulated with conventional biologically active and/or inactive carriers and excipients with or without a biodegradable material, which will be selected in accord with ordinary practice. As used herein, the phrase “biologically active” refers to a characteristic of any substance that has activity in a biological system (e.g., cell culture, organism, etc.). For instance, a substance that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component polymers. In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10. The therapeutically effective amount of active ingredient can be readily determined by a skilled clinician using conventional dose escalation studies. Typically, the active ingredient will be administered in a dose from 0.01 milligrams to 2 grams. In one embodiment, the dosage will be from about 10 milligrams to 450 milligrams. In another embodiment, the dosage will be from about 25 to about 250 milligrams. In another embodiment, the dosage will be about 50 or 100 milligrams. In one embodiment, the dosage will be about 100 milligrams. It is contemplated that the active ingredient may be administered once, twice or three times a day. Also, the active ingredient may be administered once or twice a week, once every two weeks, once every three weeks, once every four weeks, once every five weeks, or once every six weeks.

The pharmaceutical composition for the active ingredient can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface-active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Formulations suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

The active ingredient can be administered by any route appropriate to the condition. Suitable routes include inhalation, oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. In one embodiment, the patient is human.

In certain embodiments, the NFG mutant, NFG^{R100W} of the present disclosure can be formulated in any suitable dosage form for an appropriate administration. In certain embodiments, provided compositions are characterized by, for example, high loading, substantially burst-free release, sustained release, and/or effective release of nucleic acid agents. By contrast, most or all currently available nucleic acid delivery systems, many of which rely on use of lipid encapsulation, and are prone to low loading efficiencies and “burst effects.”

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

In some embodiments, compositions described herein include one or more agents. Exemplary agents include, but are not limited to, small molecules (e.g. cytotoxic agents), nucleic acid (e.g., siRNA, RNAi, and microRNA agents), proteins (e.g. antibodies), peptides, lipids, carbohydrates, hormones, metals, radioactive elements and compounds, drugs, vaccines, immunological agents, etc., and/or combinations thereof.

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Materials and Methods

Animals: All animal procedures and protocols have been approved by UCSD IACUC (Protocol #(S15159).

NGF^{WT}, NGF^{R100W}: Mouse NGF was purified from the submaxillary glands of adult male mice as previously described (Wu et al., 2001; Wu et al., 2007). NGF^{R100W} was a generous gift from Dr Changping Shi of MCLAB (320 Harbor Way, South San Francisco, Calif. 94080).

Injection: Injection of either NGF^{WT}, or NGF^{R100W} was performed according to the procedure as previously published (Sung et al., 2018). Briefly, NGF^{WT} or NGF^{R100W} protein was injected into the superficial dorsal skin of one of the hindpaws.

55° C. Hotplate Pain Test: Mice were placed on a temperature (55° C.) controlled metal surface with a Plexiglas enclosure (Ugo Basile, Italy). The time (s) it takes for the mouse to respond were recorded, if the animal either licks or lifts the hind paw or tries to escape by jumping.

Structural analysis of peripheral sensory fibers: Footpad skin biopsies. Paw skins from the hind limbs were used to examine the onset and progression of distal axonal neuropathy (Dacci et al., 2010). Mice were anesthetized. Under an operating microscope, the hind paw was sterilized. A small sterilized skin punch, 1 mm in diameter, was introduced 1 mm perpendicular to the surface of pad skin, and twisted to collect a skin sample (Dacci et al., 2010; Jolivalt et al., 2012; Jolivalt et al., 2008). The small wound was closed with a suture. These paw skin biopsies were post-fixed, cryo-protected and embedded in Tissue-Tek Optimum Cutting Temperature compound (Thermo). Sections (20 μm) were cut using a cryostat (Leica) and mounted on Superfrost⁺ slides (VWR). Selected sections were perforated, blocked and followed by incubation with primary rabbit anti-PGP9.5 (1:2500, AbD Serotec), or rabbit anti-NF200 (1:1000; Sigma) overnight at 4° C. Sections were washed with PBS, incubated with appropriate Alexa-488 or Alexa 568-conjugated secondary antibodies (1:500; Life Technologies) for 1 h at RT. Fluorescence images were acquired with a Leica SP5 Confocal Scanning Laser Microscope. Quantitation and analysis intraepidermal nerve fiber (IENF) and sub-epidermal nerve plexus (SNP) (Jolivalt et al., 2012) were performed blindly with NIH ImageJ. The number and density of IENF or SNP were measured.

Statistical Analysis. Statistical analyses were carried out using GraphPad Prism (GraphPad Software, Inc, La Jolla, Calif.). For two group comparisons, Student's t-test was performed. For multiple comparisons, one-way ANOVA or Two-way ANOVA with appropriate post-hoc tests were adopted. All data are represented as mean values ±SEM. p<0.05 is considered statistically significant; p<0.01 will be highly significant.

### Example 2

### NGF^{R100W} Protein can be Administered at 10× of NGF^{WT} without Inducing Significant Pain in Wildtype Adult Mice

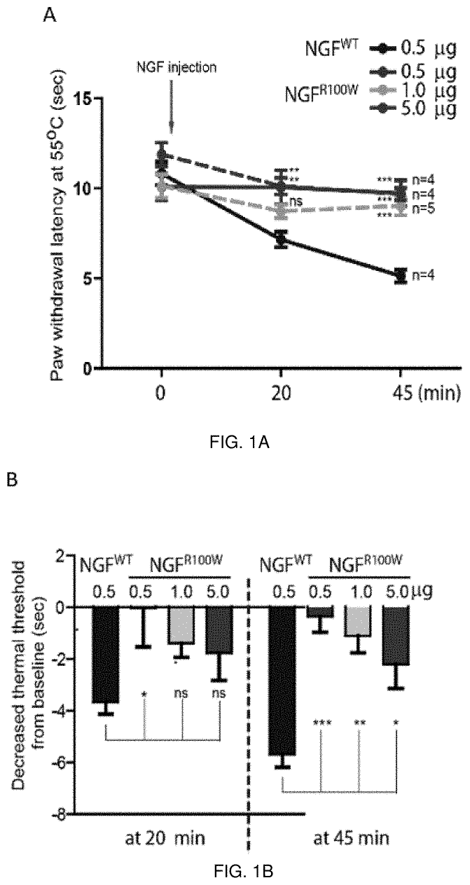

To define the dose response cure of adult mice to pain induced by NGF^{R100W}, intraplantar injection of NGF^{R100W} (0.5, 1.0, 5.0 μg) was performed and compared to NGF^{WT} (0.5 μg). The baseline response was measured (0). Adult WT mice (3 months old) were injected with 0.5 μg NGF^{WT} (n=4) or NGF^{R100W} at 0.5 μg (n=4), 1.0 μg (n=5), 5.0 μg (n=4) at indicated doses and hotplate (55° C.) assays were performed 20 min and 45 min after injection. Paw withdraw latencies (sec) were recorded (FIG. 1A). In FIG. 1B, decreased thermal thresholds were presented. The results demonstrated that injection of NGF^{R100W} even at 5.0 μg did not induce significant sensitization in the wildtype mice.

### Example 3

### Generation of a Knockin Mouse Model of Charcot Marie Tooth Peripheral Sensory Neuropathy 2B (CMT2B)

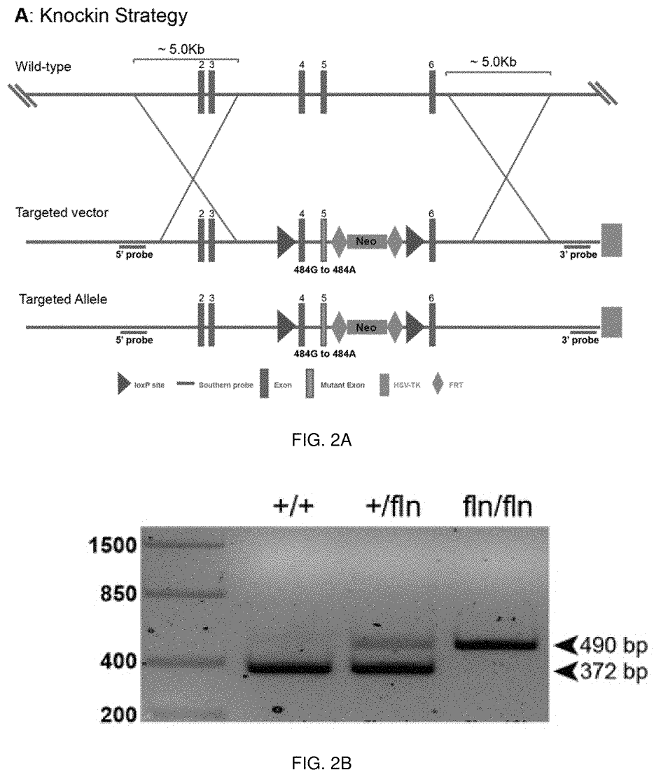

A knockin mouse model (C57BL6) for CMT2B was made by changing 484G to A (V1 62>M) in Rab7 Exon 5 (FIG. 2A). The mutated allele was introduced into the mouse genome, together with the selection marker NeoR and two LoxP sites. The LoxP sites facilitate studies in which selective deletion of the mutated allele is needed using Cre. Genotyping was performed using the PCR primer pair (FIG. 2B). The wt gives rise to a 373 bp fragment while the mutant allele (Rab7V162M:Neo) produces a 490 bp. All three genotypes: wt (+/+), heterozygote (fln/+) and homozygote (fln/fln) with a typical Mendelian segregation ratio were obtained. Both the fln/+ and fln/fln pubs survive to full adulthood.

### Example 4

### The CMT2B Mutant Mice Show Significant Reduction in Small Sensory Fibers

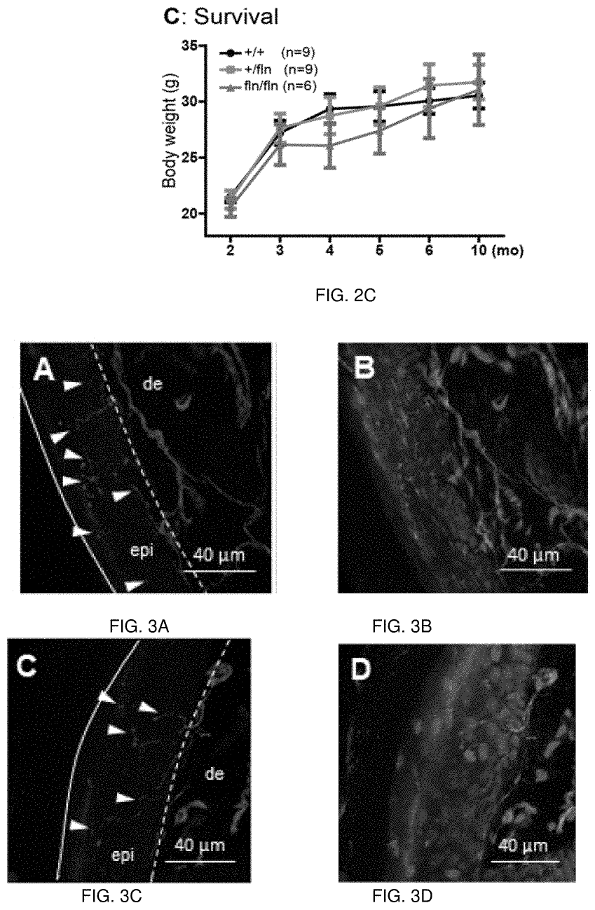

To assess the neuropathy phenotype in the CMT2B Rab7^{V162M} mutant mice, cutaneous innervation of sensory intraepidermal nerve fibers (IENFs) were compared in footpad skin biopsies from 9 months wild-type, heterozygous, and homozygous genotypes (FIGS. 3A-3G). IENFs were immunostained for PGP 9.5, a ubiquitin hydrolase and pan-neuronal marker. Samples from three different mice of each genotype were analyzed (n=3). Only IENF crossing the dermal-epidermal junction were quantified. The signal intensity of IENF profiles was normalized to epidermal area to account for variations in epidermis thickness. A decreased density of PGP 9.5 fibers in the epidermis was expected in disease phenotypes compared to the wild-type control. Indeed, immunostaining for both disease genotypes appeared to demonstrate severe small fiber neuropathy compared to the control (FIG. 3A-3F).

Strong IENF signal intensities and extensive secondary branching were apparent in wild-type controls, verifying that the staining protocol had worked (FIGS. 3A & 3B). Rab7^{V162M} heterozygous mice showed reduced secondary branching and fewer PGP 9.5 signals in the epidermis, although some nerve structures were apparent (FIGS. 3C & 3D). In Rab7V162M homozygotes, no secondary branching was observed, and PGP-9.5 signals were scarce and localized to only a few regions on each skin sample (FIGS. 3E & 3F). A significant variation in PGP 9.5 signal intensity of stained IENF fibers was confirmed in all three groups, with a two-fold difference observed in the heterozygous genotype and a striking ten-fold difference in the homozygous genotype, compared to the wild-type control (FIG. 3G). These results are evidence that the CMT2B mutant mice develop significant loss of small sensory fibers at 9 months of age.

### Example 5

### NGF^{R100W} Rescued Sensory Functional Degeneration in CMT2B Mutant Mice

Preclinical studies of NGF^{R100W} were performed. The mouse knockin model of CMT2B peripheral sensory neuropathy was used. The disease is caused by missense mutation(s) in Rab7 GTPase. Patients suffer from loss of severe pain sensation that frequently leads to amputation. CMT2B is believed to be an axonal length-dependent disease: i.e. neurons with the longest axons are affected first. It is demonstrated that NGF/TrkA signaling is impaired in sensory neurons that may account for the structural and functional degeneration of sensory fibers in CMT2B. A knockin mouse model was generated and it has shown the mutant mice developed significant peripheral sensory deficits at 9 months of age.

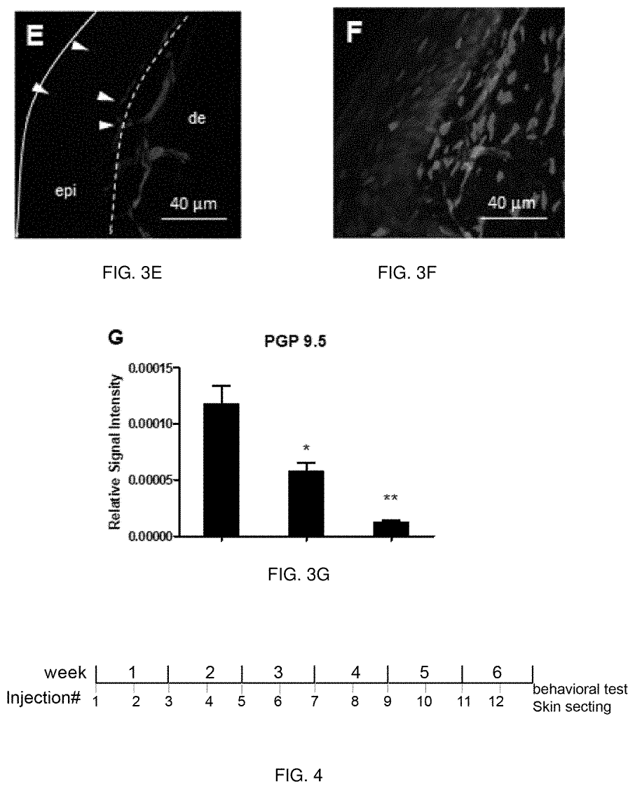

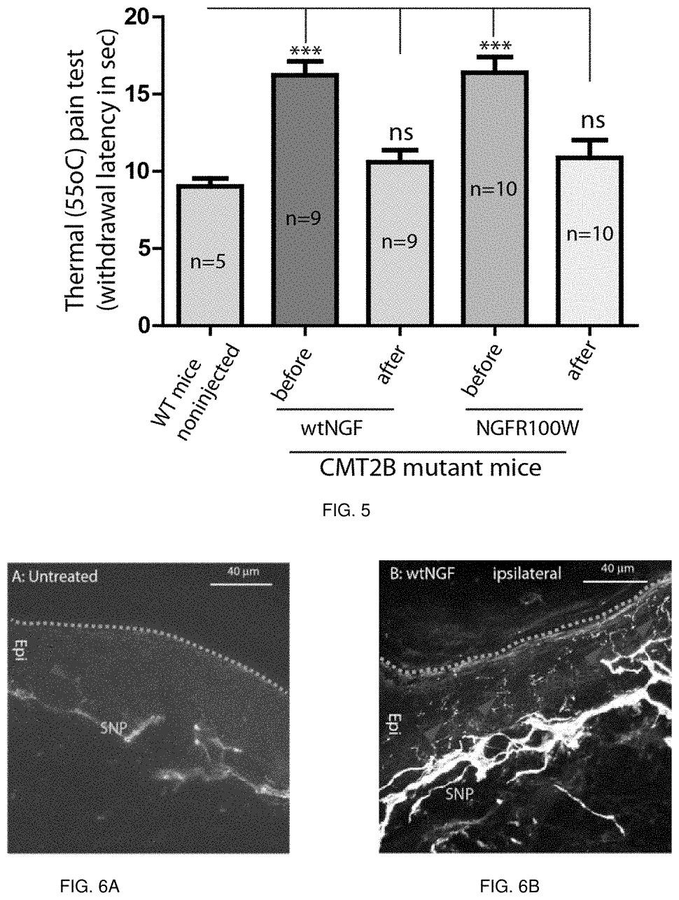

To test if NGF^{R100W} was effective in rescuing nociception in CMT2B mutant mice, 1 μg NGF^{R100W} or 1 μg NGF^{WT} was injected intradermally into 9 months old CMT2B mutant mice (twice a week for a total of 6 weeks) following by hot plate nociceptive behavior test (FIG. 4). Briefly, the result shown in FIG. 5 indicates that injection of NGF^{R100W} was as effective as wtNGF in restoring pain sensation in the mutant mice (FIG. 5). Statistics showed there was no significant difference in the efficacy between NGF^{R100W} and wtNGF.

### Example 6

### NGF^{R100W} Rescued Sensory Structural Degeneration in CMT2B Mutant Mice

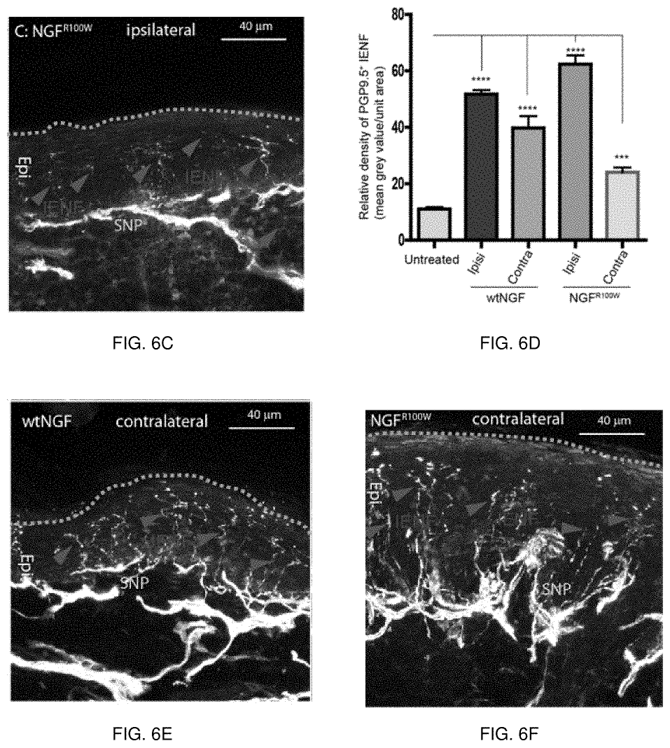

To test if NGF^{R100W} was effective in rescuing sensory fiber degeneration in CMT2B mutant mice, immunohistochemistry was performed and the analysis on intra-epidermal nerve fibers (IENFs) in the hind paw skin was completed. Specifically, the skin samples were sectioned and stained with a specific antibody against PGP9.5 to stain and quantitate IENFs (arrow heads in FIGS. 6A-6F). As shown in FIGS. 6A-6F, in 9 months old CMT2B mutant mice, IENFs were rarely detected (FIG. 6A). Mice treated with either wtNGF (FIG. 6B) or NGF^{R100W} (FIG. 6C) showed significant recovery in IENFs in their hind paw skin. This is evident in quantification of IENF density (FIG. 6D). A very interesting finding is that IENFs were also significantly recovered in the contralateral side apparently more than the ipsilateral side (top images versus bottom images in FIGS. 6B and 6C, also 6D). These data may suggest that repeated needle injection onto the skin and the volume of injected liquid might induce minor damage in the tissue, which consequently could interfere neuronal recovery. Nevertheless, these data have demonstrated that NGF^{R100W} injection successfully stimulated the regeneration of IENFs not only in ipsilateral but also contralateral paws. Taken together, these data support that NGF^{R100W} promotes effective regeneration of peripheral sensory neurons both structurally and functionally.

In summary, the Examples presented herein demonstrates that NGF^{R100W} can be injected at 10× of normal wildtype NGF without inducing significant sensitization in vivo, strongly indicating that NGF^{R100W} do not elicit strong pain effect in clinical testing. Further, these Examples provide evidence suggesting that NGF^{R100W} is as effective in promoting the regrowth of small sensory fibers and rescuing functional degeneration of peripheral sensory neuropathy. These preclinical studies provide support for the translational potential of NGF^{R100W} as a novel therapy for treating peripheral neuropathy.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

Aloe, L., Rocco, M. L., Bianchi, P., and Manni, L. (2012). Nerve growth factor: from the early discoveries to the potential clinical use. Journal of Translational Medicine 10, 239.
Apfel, S. C. (1999). Neurotrophic factors in the therapy of diabetic neuropathy. Am J Med 107, 34S-42S.
Apfel, S. C. (2002). Nerve growth factor for the treatment of diabetic neuropathy: what went wrong, what went right, and what does the future hold? Int Rev Neurobiol 50, 393-413.
Apfel, S. C., and Kessler, J. A. (1996). Neurotrophic factors in the treatment of peripheral neuropathy. Ciba Found Symp 196, 98-108; discussion 108-112.
Apfel, S. C., Kessler, J. A., Adornato, B. T., Litchy, W. J., Sanders, C., and Rask, C. A. (1998). Recombinant human nerve growth factor in the treatment of diabetic polyneuropathy. NGF Study Group. Neurology 51, 695-702.
Bartus, R., Dean, R., Beer, B., and Lippa, A. (1982). The cholinergic hypothesis of geriatric memory dysfunction. Science 217, 408-414.
Capsoni, S., Ugolini, G., Comparini, A., Ruberti, F., Berardi, N., and Cattaneo, A. (2000). Alzheimer-like neurodegeneration in aged antinerve growth factor transgenic mice. Proc Natl Acad Sci USA 97, 6826-6831.
Carvalho, O. P., Thornton, G. K., Hertecant, J., Houlden, H., Nicholas, A. K., Cox, J. J., Rielly, M., AI-Gazali, L., and Woods, C. G. (2011). A novel NGF mutation clarifies the molecular mechanism and extends the phenotypic spectrum of the HSAN5 neuropathy. J Med Genet 48, 131-135.
Cattaneo, A., and Calissano, P. (2012). Nerve growth factor and Alzheimer's disease: new facts for an old hypothesis. Mol Neurobiol 46, 588-604.
Chao, M. V. (2003). Neurotrophins and their receptors: a convergence point for many signalling pathways. Nat Rev Neurosci 4, 299-309.
Chao, M. V., and Bothwell, M. (2002). Neurotrophins: to cleave or not to cleave. Neuron 33, 9-12.
Chao, M. V., and Hempstead, B. L. (1995). p75 and Trk: a two-receptor system. Trends Neurosci 18, 321-326.
Chen, L. W., Yung, K. K., Chan, Y. S., Shum, D. K., and Bolam, J. P. (2008). The proNGF-p75NTR-sortilin signalling complex as new target for the therapeutic treatment of Parkinson's disease. CNS Neurol Disord Drug Targets 7, 512-523.
Conner, J. M., Franks, K. M., Titterness, A. K., Russell, K., Merrill, D. A., Christie, B. R., Sejnowski, T. J., and Tuszynski, M. H. (2009). NGF is essential for hippocampal plasticity and learning. J Neurosci 29, 10883-10889.
Conover, J. C., and Yancopoulos, G. D. (1997). Neurotrophin regulation of the developing nervous system: analyses of knockout mice. Rev Neurosci 8, 13-27.
Cooper, J. D., Salehi, A., Delcroix, J. D., Howe, C. L., Belichenko, P. V., Chua-Couzens, J., Kilbridge, J. F., Carlson, E. J., Epstein, C. J., and Mobley, W. C. (2001). Failed retrograde transport of NGF in a mouse model of Down's syndrome: reversal of cholinergic neurodegenerative phenotypes following NGF infusion. Proceedings of the National Academy of Sciences of the United States of America 98, 10439-10444.
Counts, S. E., and Mufson, E. J. (2005). The role of nerve growth factor receptors in cholinergic basal forebrain degeneration in prodromal Alzheimer disease. J Neuropathol Exp Neurol 64, 263-272.
Cuello, A. C., Bruno, M. A., and Bell, K. F. (2007). NGF-cholinergic dependency in brain aging, MCI and Alzheimer's disease. Curr Alzheimer Res 4, 351-358.
Dacci, P., Dina, G., Cerri, F., Previtali, S. C., Lopez, I. D., Lauria, G., Feltri, M. L., Bolino, A., Comi, G., Wrabetz, L., et al. (2010). Foot pad skin biopsy in mouse models of hereditary neuropathy. Glia 58, 2005-2016.
Davies, P., and Maloney, A. J. (1976). Selective loss of central cholinergic neurons in Alzheimer's disease. Lancet 2, 1403.
Deiana, S., Platt, B., and Riedel, G. (2011). The cholinergic system and spatial learning. Behav Brain Res 221, 389-411.
Delcroix, J. D., Valletta, J., Wu, C., Howe, C. L., Lai, C. F., Cooper, J. D., Belichenko, P. V., Salehi, A., and Mobley, W. C. (2004). Trafficking the NGF signal: implications for normal and degenerating neurons. Prog Brain Res 146, 3-23.
Doody, R. S. (2003). Current treatments for Alzheimer's disease: cholinesterase inhibitors. J Clin Psychiatry 64 Suppl 9, 11-17.
Drachman, D. A., and Leavitt, J. (1974). Human memory and the cholinergic system. A relationship to aging? Arch Neurol 30, 113-121.
Dutar, P., Bassant, M. H., Senut, M. C., and Lamour, Y. (1995). The septohippocampal pathway: structure and function of a central cholinergic system. Physiological reviews 75, 393-427.
Einarsdottir, E., Carlsson, A., Minde, J., Toolanen, G., Svensson, O., Solders, G., Holmgren, G., Holmberg, D., and Holmberg, M. (2004a). A mutation in the nerve growth factor beta gene (NGFB) causes loss of pain perception. Human molecular genetics 13, 799-805.
Einarsdottir, E., Carlsson, A., Minde, J., Toolanen, G., Svensson, O., Solders, G., Holmgren, G., Holmberg, D., and Holmberg, M. (2004b). A mutation in the nerve growth factor beta gene (NGFB) causes loss of pain perception. Hum Mol Genet 13, 799-805.
Enocson, A. G., Minde, J., and Svensson, O. (2006). Socket wall addition device in the treatment of recurrent hip prosthesis dislocation: good outcome in 12 patients followed for 4.5 (1-9) years. Acta Orthop 77, 87-91.
Eriksdotter Jonhagen, M., Nordberg, A., Amberla, K., Backman, L., Ebendal, T., Meyerson, B., Olson, L., Seiger, Shigeta, M., Theodorsson, E., et aL. (1998). Intracerebroventricular infusion of nerve growth factor in three patients with Alzheimer's disease. Dement Geriatr Cogn Disord 9, 246-257.
Everitt, B. J., and Robbins, T. W. (1997). Central cholinergic systems and cognition. Annu Rev Psychol 48, 649-684.
Fischer, W., Wictorin, K., Bjorklund, A., Williams, L. R., Varon, S., and Gage, F. H. (1987). Amelioration of cholinergic neuron atrophy and spatial memory impairment in aged rats by nerve growth factor. Nature 329, 65-68.
Francis, P., Palmer, A., Snape, M., and Wilcock, G. (1999). The cholinergic hypothesis of Alzheimer's disease: a review of progress. Journal of Neurology, Neurosurgery, and Psychiatry 66, 137-147.
Gnahn, H., Hefti, F., Heumann, R., Schwab, M. E., and Thoenen, H. (1983). NGF-mediated increase of choline acetyltransferase (ChAT) in the neonatal rat forebrain: evidence for a physiological role of NGF in the brain? Brain Res 285, 45-52.
Grothe, M., Heinsen, H., and Teipel, S. J. (2012). Atrophy of the cholinergic Basal forebrain over the adult age range and in early stages of Alzheimer's disease. Biol Psychiatry 71, 805-813.
Grutzendler, J., and Morris, J. C. (2001). Cholinesterase inhibitors for Alzheimer's disease. Drugs 61, 41-52.
Hellweg, R., and Hartung, H. D. (1990). Endogenous levels of nerve growth factor (NGF) are altered in experimental diabetes mellitus: a possible role for NGF in the pathogenesis of diabetic neuropathy. J Neurosci Res 26, 258-267.
Holtzman, D. M., Li, Y., Parada, L. F., Kinsman, S., Chen, C. K., Valletta, J. S., Zhou, J., Long, J. B., and Mobley, W. C. (1992). p140trk mRNA marks NGF-responsive forebrain neurons: evidence that trk gene expression is induced by NGF. Neuron 9, 465-478.
Houeland, G., Romani, A., Marchetti, C., Amato, G., Capsoni, S., Cattaneo, A., and Marie, H. (2010). Transgenic mice with chronic NGF deprivation and Alzheimer's disease-like pathology display hippocampal region-specific impairments in short- and long-term plasticities. J Neurosci 30, 13089-13094.
Huang, E. J., and Reichardt, L. F. (2001). Neurotrophins: Roles in Neuronal Development and Function. Annual review of neuroscience 24, 677-736.
Jakobsen, J., Brimijoin, S., Skau, K., Sidenius, P., and Wells, D. (1981). Retrograde axonal transport of transmitter enzymes, fucose-labeled protein, and nerve growth factor in streptozotocin-diabetic rats. Diabetes 30, 797-803.
Jeon, O., Powell, C., Solorio, L. D., Krebs, M. D., and Alsberg, E. (2011). Affinity-based growth factor delivery using biodegradable, photocrosslinked heparin-alginate hydrogels. Journal of controlled release: official journal of the Controlled Release Society 154, 258-266.
Jha, A. K., Mathur, A., Svedlund, F. L., Ye, J., Yeghiazarians, Y., and Healy, K. E. (2015). Molecular weight and concentration of heparin in hyaluronic acid-based matrices modulates growth factor retention kinetics and stem cell fate. Journal of controlled release: official journal of the Controlled Release Society 209, 308-316.
Jolivalt, C. G., Calcutt, N. A., and Masliah, E. (2012). Similar pattern of peripheral neuropathy in mouse models of type 1 diabetes and Alzheimer's disease. Neuroscience 202, 405-412.
Jolivalt, C. G., Lee, C. A., Beiswenger, K. K., Smith, J. L., Orlov, M., Torrance, M. A., and Masliah, E. (2008). Defective insulin signaling pathway and increased glycogen synthase kinase-3 activity in the brain of diabetic mice: parallels with Alzheimer's disease and correction by insulin. J Neurosci Res 86, 3265-3274.
Kew, J. N., Smith, D. W., and Sofroniew, M. V. (1996). Nerve growth factor withdrawal induces the apoptotic death of developing septal cholinergic neurons in vitro: protection by cyclic AMP analogue and high potassium. Neuroscience 70, 329-339.
Latina, V., Caioli, S., Zona, C., Ciotti, M. T., Amadoro, G., and Calissano, P. (2017). Impaired NGF/TrkA Signaling Causes Early AD-Linked Presynaptic Dysfunction in Cholinergic Primary Neurons. Frontiers in cellular neuroscience 11, 68.
Lehmann, M., Fournier, A., Selles-Navarro, I., Dergham, P., Sebok, A., Leclerc, N., Tigyi, G., and McKerracher, L. (1999). Inactivation of Rho signaling pathway promotes CNS axon regeneration. J Neurosci 19, 7537-7547.
Levi-Montalcini, R. (1987). The nerve growth factor 35 years later. Science 237, 1154-1162.
Levi-Montalcini, R., and Hamburger, V. (1951). Selective growth stimulating effects of mouse sarcoma on the sensory and sympathetic nervous system of the chick embryo. J Exp Zool 116, 321-361.
Li, X., and Jope, R. S. (1995). Selective inhibition of the expression of signal transduction proteins by lithium in nerve growth factor-differentiated PC12 cells. J Neurochem 65, 2500-2508.
McConoughey, S. J., Howlin, R. P., Wiseman, J., Stoodley, P., and Calhoun, J. H. (2015). Comparing PMMA and calcium sulfate as carriers for the local delivery of antibiotics to infected surgical sites. J Biomed Mater Res B Appl Biomater 103, 870-877.
Minde, J., Andersson, T., Fulford, M., Aguirre, M., Nennesmo, I., Remahl, I. N., Svensson, O., Holmberg, M., Toolanen, G., and Solders, G. (2009). A novel NGFB point mutation: a phenotype study of heterozygous patients. Journal of neurology, neurosurgery, and psychiatry 80, 188-195.
Minde, J., Svensson, O., Holmberg, M., Solders, G., and Toolanen, G. (2006). Orthopedic aspects of familial insensitivity to pain due to a novel nerve growth factor beta mutation. Acta Orthop 77, 198-202.
Minde, J., Toolanen, G., Andersson, T., Nennesmo, I., Remahl, I. N., Svensson, O., and Solders, G. (2004). Familial insensitivity to pain (HSAN V) and a mutation in the NGFB gene. A neurophysiological and pathological study. Muscle Nerve 30, 752-760.
Minde, J. K. (2006). Norrbottnian congenital insensitivity to pain. Acta Orthop Suppl 77, 2-32.
Morrison, I., Loken, L. S., Minde, J., Wessberg, J., Perini, I., Nennesmo, I., and Olausson, H. (2011). Reduced C-afferent fibre density affects perceived pleasantness and empathy for touch. Brain 134, 1116-1126.
Mufson, E. J., Conner, J. M., and Kordower, J. H. (1995). Nerve growth factor in Alzheimer's disease: defective retrograde transport to nucleus basalis. Neuroreport 6, 1063-1066.
Niewiadomska, G., Mietelska-Porowska, A., and Mazurkiewicz, M. (2011). The cholinergic system, nerve growth factor and the cytoskeleton. Behav Brain Res 221, 515-526.
Olson, L. (1993). NGF and the treatment of Alzheimer's disease. Exp Neurol 124, 5-15.
Ordonez, G., Fernandez, A., Perez, R., and Sotelo, J. (1994). Low contents of nerve growth factor in serum and submaxillary gland of diabetic mice. A possible etiological element of diabetic neuropathy. J Neurol Sci 121, 163-166.
Pant, J., Sundaram, J., Goudie, M. J., Nguyen, D. T., and Handa, H. (2019). Antibacterial 3D bone scaffolds for tissue engineering application. J Biomed Mater Res B Appl Biomater 107, 1068-1078.
Perinelli, D. R., Fagioli, L., Campana, R., Lam, J. K. W., Baffone, W., Palmieri, G. F., Casettari, L., and Bonacucina, G. (2018). Chitosan-based nanosystems and their exploited antimicrobial activity. Eur J Pharm Sci 117, 8-20.
Perini, I., Tavakoli, M., Marshall, A., Minde, J., and Morrison, I. (2016). Rare human nerve growth factor-beta mutation reveals relationship between C-afferent density and acute pain evaluation. J Neurophysiol 116, 425-430.
Rabea, E. I., Badawy, M. E., Stevens, C. V., Smagghe, G., and Steurbaut, W. (2003). Chitosan as antimicrobial agent: applications and mode of action. Biomacromolecules 4, 1457-1465.
Rabenstein, D. L. (2002). Heparin and heparan sulfate: structure and function. Nat Prod Rep 19, 312-331.
Rindone, A. N., Kachniarz, B., Achebe, C. C., Riddle, R. C., O'Sullivan, A. N., Dorafshar, A. H., and Grayson, W. L. (2019). Heparin-Conjugated Decellularized Bone Particles Promote Enhanced Osteogenic Signaling of PDGF-BB to Adipose-Derived Stem Cells in Tissue Engineered Bone Grafts. Advanced healthcare materials 8, e1801565.
Sagafos, D., Kleggetveit, I. P., Helas, T., Schmidt, R., Minde, J., Namer, B., Schmelz, M., and Jorum, E. (2016). Single-Fiber Recordings of Nociceptive Fibers in Patients With HSAN Type V With Congenital Insensitivity to Pain. Clin J Pain 32, 636-642.
Salehi, A., Delcroix, J. D., and Swaab, D. F. (2004). Alzheimer's disease and NGF signaling. J Neural Transm (Vienna) 111, 323-345.
Schliebs, R., and Arendt, T. (2006). The significance of the cholinergic system in the brain during aging and in Alzheimer's disease. J Neural Transm (Vienna) 113, 1625-1644.
Schmitz, T. W., and Nathan Spreng, R. (2016). Basal forebrain degeneration precedes and predicts the cortical spread of Alzheimer's pathology. Nat Commun 7, 13249.
Shaikh, S. S., Nahorski, M. S., and Woods, C. G. (2018). A third HSAN5 mutation disrupts the nerve growth factor furin cleavage site. Mol Pain 14, 1744806918809223.
Sofroniew, M. V., Cooper, J. D., Svendsen, C. N., Crossman, P., Ip, N. Y., Lindsay, R. M., Zafra, F., and Lindholm, D. (1993). Atrophy but not death of adult septal cholinergic neurons after ablation of target capacity to produce mRNAs for NGF, BDNF, and NT3. The Journal of neuroscience: the official journal of the Society for Neuroscience 13, 5263-5276.
Sung, K., Ferrari, L. F., Yang, W., Chung, C., Zhao, X., Gu, Y., Lin, S., Zhang, K., Cui, B., Pearn, M. L., et aL. (2018). Swedish Nerve Growth Factor Mutation (NGF(R100W)) Defines a Role for TrkA and p75(NTR) in Nociception. J Neurosci 38, 3394-3413.
Svendsen, C. N., Kew, J. N., Staley, K., and Sofroniew, M. V. (1994). Death of developing septal cholinergic neurons following NGF withdrawal in vitro: protection by protein synthesis inhibition. J Neurosci 14, 75-87.
Teipel, S., Heinsen, H., Amaro, E., Jr., Grinberg, L. T., Krause, B., and Grothe, M. (2014). Cholinergic basal forebrain atrophy predicts amyloid burden in Alzheimer's disease. Neurobiology of Aging 35, 482-491.
Tuszynski, M. H., and Gage, F. H. (1995). Bridging grafts and transient nerve growth factor infusions promote long-term central nervous system neuronal rescue and partial functional recovery. Proc Natl Acad Sci USA 92, 4621-4625.
Tuszynski, M. H., Thal, L., Pay, M., Salmon, D. P., U, H. S., Bakay, R., Patel, P., Blesch, A., Vahlsing, H. L., Ho, G., et aL. (2005). A phase 1 clinical trial of nerve growth factor gene therapy for Alzheimer disease. Nat Med 11, 551-555.
Tuszynski, M. H., Yang, J. H., Barba, D., U, H. S., Bakay, R. A., Pay, M. M., Masliah, E., Conner, J. M., Kobalka, P., Roy, S., et aL. (2015). Nerve Growth Factor Gene Therapy: Activation of Neuronal Responses in Alzheimer Disease. JAMA Neurol 72, 1139-1147.
Ward, N. L., Stanford, L. E., Brown, R. E., and Hagg, T. (2000). Cholinergic medial septum neurons do not degenerate in aged 129/Sv control or p75(NGFR)−/−mice. Neurobiol Aging 21, 125-134.
Whitehouse, P., Price, D., Struble, R., Clark, A., Coyle, J., and Delon, M. (1982). Alzheimer's disease and senile dementia: loss of neurons in the basal forebrain. Science 215, 1237-1239.
Wilkinson, D. G., Francis, P. T., Schwam, E., and Payne-Parrish, J. (2004). Cholinesterase inhibitors used in the treatment of Alzheimer's disease: the relationship between pharmacological effects and clinical efficacy. Drugs Aging 21, 453-478.
Woodruff, M. A., and Hutmacher, D. W. (2010). The return of a forgotten polymer—Polycaprolactone in the 21 st century, Vol 35 (Elseiveir).
Wu, C., Lai, C. F., and Mobley, W. C. (2001). Nerve growth factor activates persistent Rap1 signaling in endosomes. J Neurosci 21, 5406-5416.
Wu, C., Ramirez, A., Cui, B., Ding, J., Delcroix, J. D., Valletta, J. S., Liu, J. J., Yang, Y., Chu, S., and Mobley, W. C. (2007). A functional dynein-microtubule network is required for NGF signaling through the Rap1/MAPK pathway. Traffic 8, 1503-1520.
Xu, X., Jha, A. K., Duncan, R. L., and Jia, X. (2011). Heparin-decorated, hyaluronic acid-based hydrogel particles for the controlled release of bone morphogenetic protein 2. Acta biomaterialia 7, 3050-3059.
Xue, J., Feng, B., Zheng, R., Lu, Y., Zhou, G., Liu, W., Cao, Y., Zhang, Y., and Zhang, W. J. (2013). Engineering ear-shaped cartilage using electrospun fibrous membranes of gelatin/polycaprolactone. Biomaterials 34, 2624-2631.
Zamecnik, C. R., Lowe, M. M., Patterson, D. M., Rosenblum, M. D., and Desai, T. A. (2017). Injectable Polymeric Cytokine-Binding Nanowires Are Effective Tissue-Specific Immunomodulators. ACS Nano 11, 11433-11440.
Zhang, K., Huang, D., Yan, Z., and Wang, C. (2017). Heparin/collagen encapsulating nerve growth factor multilayers coated aligned PLLA nanofibrous scaffolds for nerve tissue engineering. Journal of biomedical materials research Part A 105, 1900-1910.

1. A method for treating a neuropathy comprising administering to a subject in need an effective amount of a composition comprising an effective amount of a NGF mutant that does not elicit pain.

2. The method of claim 1, wherein the NGF mutant is NGF^{R100W}.

3. The method of claim 1, where said composition is micro-rods that deliver the NGF mutant.

4. The method of claim 1, wherein said composition is nano-rods that deliver the NGF mutant.

5. The method of claim 4, wherein said nano-rods are poly-caprolactone (PCL) nanowire platform functionalized for bioactivity through the attachment of NGF using a layer-by-layer (LbL) electrostatic assembly approach.

6. The method of claim 1, where said neuropathy is caused by diabetes, HIV, Chemotherapy, infections, injuries, or exposure to a toxin.

7. The method of claim 1, wherein said subject in need is a human being who suffers with neuropathy.

8. The method of claim 1, wherein said subject in need is a human being who suffers pain induced by a treatment with NGF for an underline disease or condition.

9. A method of treating or preventing pain induced by a treatment with a NGF for an underline disease or condition, comprising administering to a subject in need an effective amount of a composition comprising an effective amount of a NGF mutant that does not elicit pain, alone or in conjunction with said NGF treatment.

10. The method of claim 9, wherein the NGF mutant is NGF^{R100W}.

11. The method of claim 9, wherein the effective amount of NGF mutant is 2-10 times more than the effective amount of NGF for the underline disease or condition.

12. The method of claim 9, where said composition is micro-rods that deliver the NGF mutant.

13. The method of claim 9, wherein said composition is nano-rods that deliver the NGF mutant.

14. The method of claim 13, wherein said nano-rods are poly-caprolactone (PCL) nanowire platform functionalized for bioactivity through the attachment of NGF using a layer-by-layer (LbL) electrostatic assembly approach.

15. A composition comprising a NGF mutant for treating a condition, wherein said NGF mutant is NGF^{R100W} that does not elicit pain.

16. The composition of claim 15, wherein said composition is micro-rods that deliver said NGF^{R100W}.

17. The composition of claim 15, wherein said composition is nano-rods that deliver said NGF^{R100W}.

18. The composition of claim 17, wherein said nano-rods are poly-caprolactone (PCL) nanowire platform functionalized for bioactivity through the attachment of NGF using a layer-by-layer (LbL) electrostatic assembly approach.

19. The composition of claim 15, where said condition is neuropathy or pain induced by a NGF treatment for an underline disease or condition.

20. The composition of claim 19, where said neuropathy is caused by diabetes, HIV, Chemotherapy, infections, injuries, or exposure to a toxin.

21-22. (canceled)
